(19) United States
(12) Patent Application Publication
(10) Pub. No.: US 2015/0098979 A1
(43) Pub. Date: Apr. 9, 2015

(54) METHOD FOR IMPARTING ENDURING BENEFICIAL FEATURES TO FLOCKED SURFACES

(71) Applicants: Robert R. Diakon, Atlantic Highlands, NJ (US); Raymond E. Guerry, Keyport, NJ (US)

(72) Inventors: Robert R. Diakon, Atlantic Highlands, NJ (US); Raymond E. Guerry, Keyport, NJ (US)

(21) Appl. No.: 14/047,000

(22) Filed: Oct. 6, 2013

(51) Int. Cl. A61K 9/70; B05D 1/16; A47G 25/14; B05D 1/28; B05D 3/02; B05D 5/00; B05D 1/14; B05D 1/24

(52) U.S. Cl. CPC A61K 9/70; B05D 1/14; B05D 1/16; B05D 1/24; B05D 1/28; B05D 3/0254; B05D 5/00; A47G 25/14
USPC 424/443; 427/180; 427/185; 427/427.4; 427/430.1; 427/429; 428/144; 428/35.7; 223/85

(57) ABSTRACT

A method of imparting enduring beneficial features to a hard flocked surface of an article is provided. The method includes encapsulating a desired beneficial agent as a core material in a polymeric microcapsule, and transferring polymeric microcapsule to the surface by a dipping, spraying, or padding application. The beneficial agent, after getting transferred to the surface of a hard flocked article, imparts long lasting performance, provides fragrance and may protect an article from odor, insect, pest, fungi, bacteria, viruses, and the like. The polymeric microcapsules deliver long lasting benefits to surfaces with flocking, such as clothes hangers. Also described is a process for manufacture of flocked items with benefit delivering polymer microcapsules, without the need for binders or adhesives. Benefit agents are released with shear force on the flocked surface, so they are released when used and provide long-lasting performance.

### BACKGROUND

1. Field of the Invention

Embodiments of the present invention relate to a method of imparting beneficial features to a flocked surface. More particularly, embodiments of the present invention relates to a method of delivering long lasting release of benefit agents to flocked surfaces, using polymeric microcapsules.

2. Description of Related Art

Polymer microcapsules are used to deliver fragrance and other actives in wash-off consumer products such as fabric softeners. Polymer microcapsules have also been used in printing applications on paper, to provide scent or release ink (e.g., carbonless paper). Flocked surfaces have fibers attached to a surface (typically with adhesive) to provide a tactile sensation such as a soft velvet feel or to increase friction to add grip to items. Flocked articles are increasingly used for various purposes, for example, to give an article a velour texture such articles as clothes hangers, t-shirts, wallpaper, gift, jewelry boxes, or upholstery. It would be difficult to print on flocked surfaces, and these surfaces cannot be washed.

Microcapsules are understood to be spherical aggregates with a diameter of about 0.001 mm to about 0.5 mm, and contain at least one solid or liquid core surrounded by at least one polymeric shell. The membrane may consist of natural, semi-synthetic or synthetic materials. The application of micro-encapsulation techniques has offered possibility of producing novel products with many beneficial features and improvements. The microcapsules can introduce important new qualities to garments and fabrics, such as fabrics with durable fragrances, odor elimination, insect repellant and insecticide, care for fabric, skin or hair (e.g., aloe), UV-ray absorbing microcapsules, thermo-changeable dyes, thermo-regulation phase change ingredients, and antimicrobials.

It is desirable to deliver fragrance benefits on flocked surfaces, to deliver these benefits in a closet, drawer or hanger where they are used. Clothes, especially jackets or coats kept in a closet, drawer or hanger, are frequently contaminated by different kinds of odors, such as smoke, sweat, drink, food or dirt. Clothes also get colonized by fungus, bacteria and infested with insects (e.g., moths). This is undesired and therefore, there is a need of method to prevent such contaminations. Use of polymeric microcapsules containing a perfume and other agents offer a probable solution, but there are problems associated therewith.

Thus, there is a need for a method for the application of a beneficial agent comprising microcapsules, on to a hard flocked surface that leads to relatively long lasting beneficial effects.

### SUMMARY

Embodiments in accordance with the present invention provide a method of imparting beneficial features to a hard flocked surface using polymeric microcapsules containing a desired beneficial agent in its core. The beneficial features imparted to the hard flocked surface comprise relatively long lasting fragrance or protection from bad odor, insects, pests, fungi, bacteria and viruses.

Embodiments in accordance with the present invention further provide polymeric microcapsules encapsulating a desired beneficial agent as a core material in a polymeric microcapsule.

Embodiments in accordance with the present invention further provide a clothes hanger, a closet or a drawer, which releases fragrance for a relatively longer time and protects a stored article from odor, insect, pest, fungi, bacteria, virus, and the like.

Embodiments in accordance with the present invention further provide a slurry mixture used for transferring microcapsules having a polymeric capsule containing a beneficial agent. Other embodiments include a wetting agent and a chemical scavenger.

Embodiments in accordance with the present invention further relate to hard flocked products including clothes hanger, closet, drawers, and/or treatment compositions comprising such microcapsules, and processes of making and using same.

Further, embodiments of the present invention can provide a number of advantages depending on its particular configuration. Embodiments of the present invention provide a method of transferring polymeric microcapsule on to a hard flocked surface, which is able to stably retain a beneficial agent for an extended period. The method is particularly suitable for imparting fragrance to a clothes hanger and the like. In addition, products having hard flocked surfaces, including closets and drawers can be enhanced using the aforementioned process. Next, the method further ensures uniform distribution of the microcapsules to avoid aesthetic defects and to provide long lasting benefits to the treated products.

These and other advantages will be apparent from the disclosure of embodiments of the present invention contained herein.

The preceding is a simplified summary of the present invention to provide an understanding of some aspects of the present invention. This summary is neither an extensive nor exhaustive overview of the present invention and its various embodiments. It is intended neither to identify key or critical elements of the present invention nor to delineate the scope of the present invention but to present selected concepts of the present invention in a simplified form as an introduction to the more detailed description presented below. As will be appreciated, other embodiments of the present invention are possible, utilizing alone or in combination, one or more of the features set forth above or described in detail below.

### BRIEF DESCRIPTION OF THE DRAWINGS

The above and still further features and advantages of the present invention will become apparent upon consideration of the following detailed description of embodiments thereof, especially when taken in conjunction with the accompanying drawing, and wherein:

FIG. 1A is a flocked cloth hanger which was not properly coated and is showing white specs;

FIG. 1B is a flocked cloth hanger which is properly coated and is showing no white specs in accordance with an embodiment of the present invention;

FIG. 2A is a schematic diagram of a method of dipping to transfer polymeric microcapsule to hard flocked surfaces in accordance with an embodiment of the present invention;

FIG. 2B is a schematic diagram of a method of spraying to transfer polymeric microcapsule to hard flocked surfaces in accordance with an embodiment of the present invention; and

FIG. 2C is a schematic diagram of a method of padding to transfer polymeric microcapsule to hard flocked surfaces in accordance with an embodiment of the present invention.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including but not limited to. To facilitate understanding, like reference numerals have been used, where possible, to designate like elements common to the figures.

### DETAILED DESCRIPTION

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising”, “including”, and “having” can be used interchangeably.

The present invention is described in detail in the following section with the aid of embodiments and the figure, which are intended to illustrate the present invention. An embodiment of the present invention provides a method of imparting beneficial features to a hard flocked surface of an article. The method includes encapsulating a desired beneficial agent as a core material in a polymeric microcapsule, and transferring polymeric microcapsule to the surface by dip, spray or padding application.

Density of microcapsules is typically lower than water and can aggregate at the surface of diluted suspensions. Such aggregates may result in rapid phase separation of the microcapsule from the bulk phase and consequently during spraying or dipping operations. This can create uneven application and yield unacceptable aesthetics. Such aggregates can be prevented by providing constant, high agitation during the application process. Further, the addition of aggregate inhibiting materials and/or suspension stabilizers may be employed.

Substances that can be used as suspension stabilizers include natural polysaccharides such as xanthan gum, semi-synthetic polysaccharides such as carboxymethylcellulose and hydroxypropylcellulose, synthetic polymers such as sodium polyacrylate, and microfine mineral powders such as magnesium aluminum silicate and high-purity bentonite. These substances can be used singly or as an admixture of two or more of said substances. These substances stabilize the capsule slurry by increasing its viscosity.

Flocked surfaces can be hydrophobic and may resist wetting during spraying, dipping or padding. To overcome this and assure uniform and complete application, high agitation is employed. In addition, the use of a wetting agent may be employed, to increase penetration rate. Wetting agents can be anionic or nonionic surfactants. Nonionic surfactants such as Tween 20 are preferred, due to their low foaming properties.

Substances that can be used as wetting agents are nonionic surfactants such as sorbitan monoester (tween 20), alcohol ethoxylates and alkyphenolethoxylates and alkyl polyglucosides. In addition, anionic surface active agents such as lignin sulfonate, condensation product of sodium naphthalene sulfonate and formaldehyde, polyoxyethylenealkylaryl sulfates, polyoxyethylenestyrylphenyl ether sulfate and polyoxyethylenenonyl ether sulfate. Nonionic and Anionic wetting agents can be used singly or as a mixture of these substances.

Suitable equipment for use in the processes disclosed herein may include a continuous high shear mixer, stirred tank reactors, homogenizers, turbine agitators, re-circulating pumps, paddle mixers, plough shear mixers, ribbon blenders, vertical axis granulators and drum mixers, both in batch and, where available, in continuous process configurations, spray dryers, and extruders.

Wall materials are typically encapsulating polymers that are formed around the benefit agent, providing a protective shell that protects the benefit agent, until it is released. Release is typically achieved with frictional shear force on the microcapsules, during use or handling of items which have been treated with microcapsules. Useful wall materials include materials selected from the group consisting of polyethylenes, polyamides, polystyrenes, polyisoprenes, polycarbonates, polyesters, polyacrylates, polyureas, polyurethanes, polyolefins, polysaccharides, epoxy resins, vinyl polymers, and mixtures thereof.

In an embodiment of the present invention, useful wall materials include materials that are sufficiently impervious to the core material and the materials in the environment in which the benefit agent containing delivery particle will be employed, to permit the delivery benefit to be obtained. Suitable impervious wall materials include materials selected from the group consisting of: reaction products of one or more amines with one or more aldehydes, such as urea cross-linked with formaldehyde or gluteraldehyde, melamine cross-linked with formaldehyde; gelatin-polyphosphate coacervates optionally cross-linked with gluteraldehyde; gelatin-gum Arabic coacervates; cross-linked silicone fluids; and polyamine reacted with polyisocyanates and mixtures thereof. In an embodiment, the wall material comprises melamine cross-linked with formaldehyde.

Encapsulating polymers include polymers formed from melamine-formaldehyde or urea-formaldehyde condensates, as well as similar types of aminoplasts. Additionally, capsules made via the simple or complex coacervation of gelatin are also used with the coating.

Useful core materials include perfumes, silicone oils, waxes, hydrocarbons, higher fatty acids, essential oils, lipids, skin coolants, antioxidants, glycerine, malodor reducing agents, odor-controlling materials, antistatic agents, softening agents, insect and moth repelling agents, antioxidants, sanitization agents, disinfecting agents, germ control agents, mold control agents, mildew control agents, antiviral agents, fabric refreshing agents and freshness extending agents, chlorine bleach odor control agents, and natural actives such as aloe vera, vitamin E, shea butter, cocoa butter, and the like, antibacterial actives, cationic polymers, and mixtures thereof. Useful core materials are not limited to above materials, and are listed for example purpose only.

According to an embodiment of the present invention, the perfume comprises Fresh Floral SB, Musk GL, Cedar, Marine Ozone, Fruity TA, Lav.Fresh 101 at a concentration of about 10 to 15 g/l.

Delivery of benefit agents can be achieved on flocked surfaces, with microcapsules that are friable when in the dry state. The level of benefit can be adjusted by adjusting the level of application. Further, the duration can be adjusted by adjusting the level of the application.

### Example 1

In one example, a microcapsule containing a fresh floral scent was applied, typical in fabric softeners, at 2 concentrations (10 g/1 and 20 g/l). The table 1 below shows sensory results over time. The scale ranges from 0-10 where ‘0’ is no odor detected, ‘5’ is moderate odor signal and 10 is extremely strong odor signal. Scores above ‘3’ are considered easily recognized by consumers. Hangers were rubbed twice then evaluated for odor strength.

TABLE 1
Sensory Results Over Time

| | Application | | | |
|---|---|---|---|---|
| Fragrance Type | Level | Initial | Month 6 | Month 12 |
| Fresh Floral SB | 20 g/l | 8.5 | 8.5 | 8.0 |
| Fresh Floral SB | 10 g/l | 7.5 | 7.0 | 6.5 |

### Example 2

In another example, the applicant discovered a wide range of scented microcapsules provide an enduring benefit over a long period of time.

TABLE 2
Scented Microcapsules Sensory Results Over Time

| Fragrance Type | Application Level | Initial | Month 6 | Month 12 |
|---|---|---|---|---|
| Fresh Floral SB | 10 g/l | 7.5 | 7.0 | 6.5 |
| Musk GL | 15 g/l | 7.0 | 7.0 | 7.0 |
| Cedar | 10 g/l | 8.0 | 7.5 | 7.5 |
| Marine Ozone | 10 g/l | 7.5 | 7.5 | 7.5 |
| Fruity TA | 10 g/l | 7.0 | 6.5 | 6.0 |
| Lav. Fresh 101 | 10 g/l | 8.0 | 7.5 | N/A |

Particle composition, where the total weight of the slurry totals 100%: water is typically about 60% with about 40% solids. The particle solids range from about 2 to 20% wall material (typically about 5 to 10%) with and Inner Phase that is about 80 to 98% of the particle (typically about 90 to 95%). The Inner phase may typically contain about 0 to 30% stabilizer (such as Mineral Oil or Neobee M5), that balance being benefit agent.

TABLE 3

| | Range | Range | Typical |
|---|---|---|---|
| Water | 65 | 55 | 60 |
| Particle | 35 | 45 | 40 |
| Wall (% particle) | 2 | 20 | 5 |
| Inner Phase Stabilizer (% particle) | 0 | 30 | 20 |
| Benefit Agent (% particle) | 98 | 50 | 75 |

Benefit agents can be fragrance ingredients that provide freshness, a signature scent or signal other benefits like hygiene, comfort, etc. The fragrance ingredients can optionally be selected from the EPA 25b list of GRAS pesticides to provide additional benefits and claims. Many of these ingredients, such as cedar oil, would be desirable in a closet with flocked hangers. In addition, the benefit agent could include materials that react with certain malodor compounds to render them odorless. Finally, benefit agents could be other pesticides to kill unwanted insects (moths, lice, fleas or bedbugs), provide hygiene benefits, or prevent mold.

The encapsulating polymers could include materials selected from the group consisting of: polyethylenes, polyamides, polystyrenes, polyisoprenes, polycarbonates, polyesters, polyacrylates, polyureas, polyurethanes, polyolefins, polysaccharides, epoxy resins, vinyl polymers, and mixtures thereof. In one embodiment, useful wall materials include materials that are sufficiently impervious to the core material and the materials in the environment in which the benefit agent containing delivery particle will be employed, to permit the delivery benefit to be obtained.

Suitable impervious wall materials include materials selected from the group consisting of: reaction products of one or more amines with one or more aldehydes, such as urea cross-linked with formaldehyde or gluteraldehyde, melamine cross-linked with formaldehyde; gelatin-polyphosphate coacervates optionally cross-linked with gluteraldehyde; gelatin-gum Arabic coacervates; cross-linked silicone fluids; polyamine reacted with polyisocyanates and mixtures thereof. In one embodiment, the wall material comprises melamine cross-linked with formaldehyde.

It is important to maintain high level of agitation during the application process, in order to maintain uniform and complete application, and avoid aesthetic defects.

Microcapsules can be applied in a number of ways in accordance with embodiments of the present invention. For example, they could be sprayed onto the flocked surface, and then dried. On irregular surfaces, such as clothes hangers, care must be taken to assure complete and even application, which will likely result in over-spray. Over-spray can be collected, optionally filtered (to remove flocking fibers, and reused/re-sprayed onto subsequent hangers.

Formaldehyde Scavenging

After certain types of formaldehyde containing microcapsule wall film is formed, the free formaldehyde remaining in the solution can be reduced or removed by the addition of urea, ethylene urea, sulfites, sugars, ammonia, amines, acrylamide, acrylamide copolymers, or other chemicals which will react with formaldehyde under suitable conditions to convert the residual formaldehyde into a harmless substance.

In an embodiment of the present invention, polymeric microcapsules may be combined with a formaldehyde scavenger. Suitable formaldehyde scavengers include materials selected from the group consisting of sodium bisulfite, urea, ethylene urea, cysteine, cysteamine, lysine, glycine, serine, carnosine, histidine, glutathione, 3,4-diaminobenzoic acid, allantoin, glycouril, anthranilic acid, methyl anthranilate, methyl 4-aminobenzoate, ethyl acetoacetate, acetoacetamide, malonamide, ascorbic acid, 1,3-dihydroxyacetone dimer, biuret, oxamide, benzoguanamine, pyroglutamic acid, pyrogallol, methyl gallate, ethyl gallate, propyl gallate, triethanol amine, succinamide, thiabendazole, benzotriazol, triazole, indoline, sulfanilic acid, oxamide, sorbitol, glucose, cellulose, poly(vinyl alcohol), partially hydrolyzed poly(vinylformamide), poly(vinyl amine), poly(ethylene imine), poly(oxyalkyleneamine), poly(vinyl alcohol)-co-poly(vinyl amine), poly(4-aminostyrene), poly(l-lysine), chitosan, hexane diol, ethylenediamine-N,N′-bisacetoacetamide, N-(2-ethylhexyl)acetoacetamide, 2-benzoylacetoacetamide, N-(3-phenylpropyl)acetoacetamide, lilial, helional, melonal, triplal, 5,5-dimethyl-1,3-cyclohexanedione, 2,4-dimethyl-3-cyclohexenecarboxaldehyde, 2,2-dimethyl-1,3-dioxan-4,6-dione, 2-pentanone, dibutyl amine, triethylenetetramine, ammonium hydroxide, benzylamine, hydroxycitronellol, cyclohexanone, 2-butanone, pentane dione, dehydroacetic acid, or a mixture thereof.

Such formaldehyde scavengers are typically combined with a slurry containing said benefit agent containing delivery microcapsules, at a level, based on total slurry weight, of from about 2 wt. % to about 18 wt. %, from about 3.5 wt. % to about 14 wt. % or even from about 5 wt. % to about 13 wt. %.

Method of Application

This process enables the uniform application of microcapsules, and provides an enduring effect as illustrated previously, without the use of binding agents or adhesives which could adversely affect the feel and texture of flocked surfaces.

In an embodiment of the present invention, a first step towards transfer of the polymeric microcapsules on a hard flocked source is premixing polymeric microcapsule encapsulating a desired beneficial agent as a core material and water in 1:1 ratio in a high shear mixer for 15 minutes. This lower viscosity provides initial mixing to break aggregates making the subsequent dilutions easier to mix to a uniform suspension. Then, as a second step diluting by adding water to the premixed slurry to achieve a concentration of 10 to 20 grams of polymeric microcapsules/liter of slurry. A suspension stabilizer and/or wetting agent may be added at a concentration of 0.01 to 1.0%, at this point of time. As a third step, re-circulate the slurry for 15 minutes at a rate of 600 gph following which the flocked article is dipped into the agitated slurry, till it gets saturated with polymeric microcapsules. A final step is drying the excess water from the flocked article, using ambient or warm, dry air.

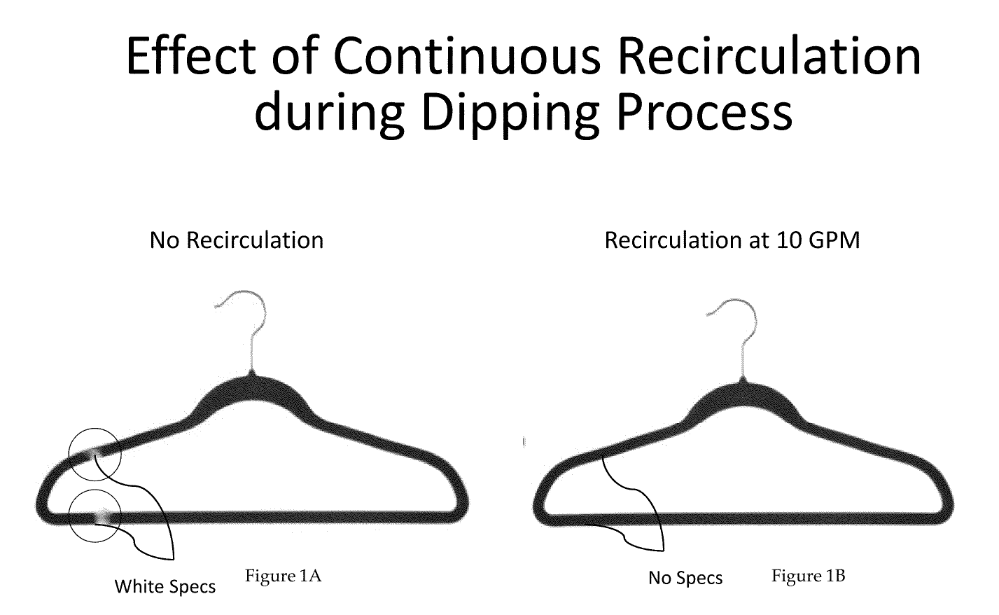

FIG. 1A is a flocked cloth hanger that was not properly coated and has unacceptable aesthetics due to white specs. FIG. 1B is a flocked cloth hanger which has been properly coated and has acceptable aesthetics due to absence of any white specs.

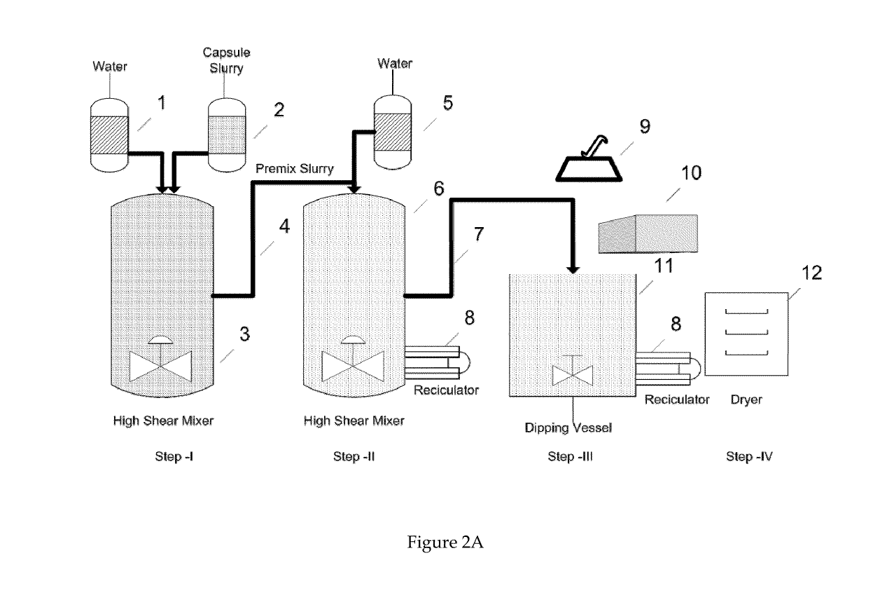

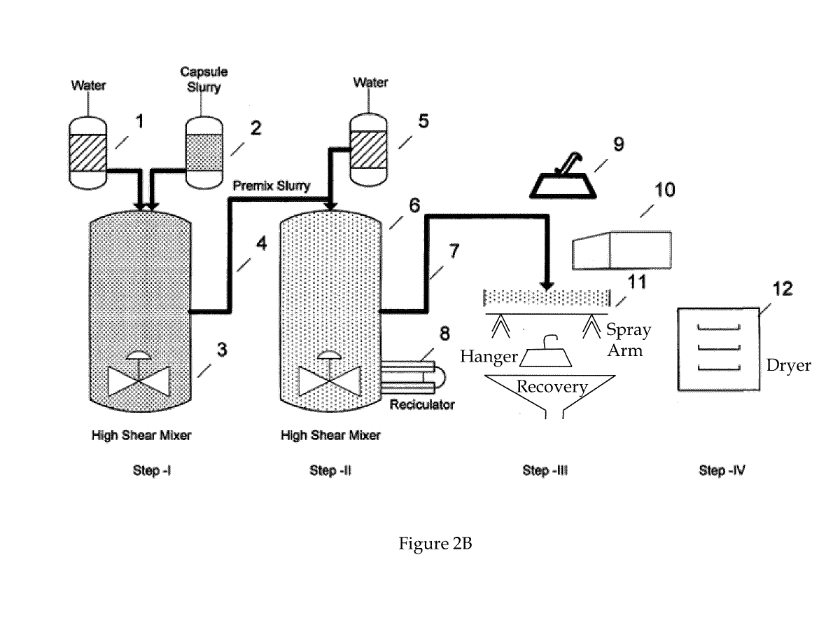

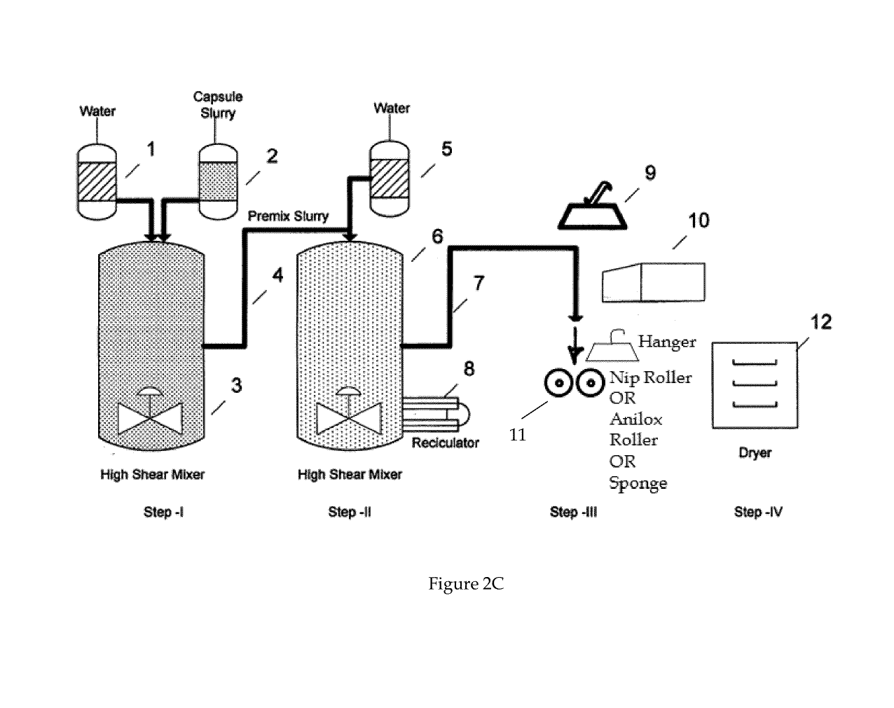

FIG. 2A is a detailed description of the dipping process in accordance with an embodiment of the present invention, described herein above, which steps and components have been given corresponding reference numerals for ease of reference. A first step towards the process of transferring polymeric microcapsules to a hard flocked surface is preparing a premix. Water 1 is mixed with Capsule slurry 2 in a high shear tank in a 1:1 ratio for 15 minutes to prepare a premix. Thereafter, in a second step the premix is transferred to a dilution tank 6 through a pipeline 4. The premix is diluted with water from tank 5 to achieve a desire dilution level preferable 10 to 20 g/l. The diluted slurry is re-circulated for 10 minutes through a slurry reservoir at a rate of 100 to 6000 gph, depending on the vessel size (600 gph in this example). Following dilution, in a third step, the diluted slurry is re-circulated to an open dipping vessel 11 through a pipeline 7, maintaining constant agitation through re-circulating at 100 to 6000 gph (600 gph in this example). Any hard flocked articles like a clothes hanger 9 or a closet item 10 can be dipped in the tank 11 for 1 second- to 10 minutes or until the article gets saturated.

According to an embodiment of the present invention, the capsule slurry suspension may be applied to flocked hangers via spraying or padding application in the third step, as long as agitation is maintained to assure consistent application.

FIG. 2B is a detailed description of the spraying process in accordance with an embodiment of the present invention, described herein above, which steps and components have been given corresponding reference numerals for ease of reference. A first step towards the process of transferring polymeric microcapsules to a hard flocked surface is preparing a premix. Water 1 is mixed with Capsule slurry 2 in a high shear tank in a 1:1 ratio for 15 minutes to prepare a premix. Thereafter, in a second step the premix is transferred to a dilution tank 6 through a pipeline 4. The premix is diluted with water from tank 5 to achieve a desire dilution level preferable 10-20 g/l. The diluted slurry is re-circulated for about 10 minutes through a slurry reservoir at a rate of 100 to 6000 gph, depending on the vessel size (600 gph in this example). Following dilution, in a third step, the diluted slurry is re-circulated to a spray arm 11 through a pipeline 7, maintaining constant agitation through re-circulating at 100 to 6000 gph (600 gph in this example). Suitable spray devices include HVLP or spray arm with a recovery tank. Spraying may be desirable for items that cannot be practically dipped.

FIG. 2C is a detailed description of the padding process in accordance with an embodiment of the present invention, described herein above, which steps and components have been given corresponding reference numerals for ease of reference. A first step towards the process of transferring polymeric microcapsules to a hard flocked surface is preparing a premix. Water 1 is mixed with Capsule slurry 2 in a high shear tank in a 1:1 ratio for 15 minutes to prepare a premix. Thereafter, in a second step the premix is transferred to a dilution tank 6 through a pipeline 4. The premix is diluted with water from tank 5 to achieve a desire dilution level preferable 10-20 g/l. The diluted slurry is re-circulated for 10 minutes through a slurry reservoir at a rate of 100 to 6000 gph, depending on the vessel size (600 gph in this example). Following dilution, in a third step, the diluted slurry is re-circulated to a padding application vessel 11 through a pipeline 7, maintaining constant agitation through re-circulating at 100-6000 gph (600 gph in this example). Padding is an application where more concentrated capsule slurry is transferred directly to a flocked surface. The advantage of this is that less water is used, which translates to less drying capacity. Padding application can be achieved with a nip roller, anilox roller or a sponge application (either automated or manually).

A low foam surfactant may be added to improve wetting, while the slurry is continuously agitated through a circulator 8 to keep the polymeric microcapsule suspended. In a final step, the coated articles are dried in ambient air or a dryer 12 to remove water.

The embodiments as disclosed and described in the application (including any accompanying claims, abstract and drawings) are intended to be illustrative and explanatory of the present invention. Modifications and variations of the disclosed embodiments, for example, of the apparatus and system employed (or to be employed) as well as the method used (or to be used), are possible; all such modifications and variations are intended to be within the scope of the present inventions.

Thus, unless expressly stated otherwise, each feature disclosed is one example only of a generic series of equivalent or similar features.

A number of variations and modifications of the present invention can be used. It would be possible to provide for some features of the present invention without providing others.

The present invention, in various embodiments, configurations, and aspects, includes components, methods, processes, systems and/or apparatus substantially as depicted and described herein, including various embodiments, sub-combinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease and\or reducing cost of implementation.

The foregoing discussion of the present invention has been presented for purposes of illustration and description. The foregoing is not intended to limit the present invention to the form or forms disclosed herein. In the foregoing Detailed Description for example, various features of the present invention are grouped together in one or more embodiments, configurations, or aspects for the purpose of streamlining the disclosure.

The features of the embodiments, configurations, or aspects of the present invention may be combined in alternate embodiments, configurations, or aspects other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate embodiment of the present invention.

Moreover, though the description of the present invention has included description of one or more embodiments, configurations, or aspects and certain variations and modifications, other variations, combinations, and modifications are within the scope of the present invention, e.g., as may be within the skill and knowledge of those in the art, after understanding the present disclosure. It is intended to obtain rights which include alternative embodiments, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of imparting beneficial and enduring features to a hard flocked surface, the method comprising:
encapsulating a desired beneficial agent as a core material in a polymeric microcapsule;
transferring the polymeric microcapsule to the surface by dip, spray or padding application under sustained agitation; and
drying the flocked surface.

2. A hard flocked article prepared in accordance with the method of claim 1.

3. A medicated hard flocked article prepared in accordance with the method of claim 1.

4. The hard flocked article of claim 2 being a clothes hanger.

5. The hard flocked article of claim 2 comprising a housing for storing clothes, woolens, leather, fabric, and perishable consumables.

6. A closet with a flocked inner surface for storing clothes, wherein the inner surface of the closet is treated in accordance with the method of claim 1.

7. The method of claim 1, wherein the desired beneficial agent is selected from a group consisting of: essential oils, perfume, insecticide, pesticide, antifungal, antibacterial, antiviral agents, insect repellent, perfume raw materials, silicone oils, waxes, malodor reducing agents, odor-controlling materials, sanitization agents, disinfecting agents, germ control agents, mold control agents, mildew control agents, antiviral agents, drying agents, stain resistance agents, fabric refreshing agents and freshness extending agents, anti-allergenic agents, skin care agents, natural actives, antibacterial actives and mixtures thereof.

8. The method of claim 1, wherein the beneficial feature is long lasting release of fragrance from the flocked article.

9. The method of claim 1, wherein the beneficial feature is release of fragrance from hard flocked surfaces when clothes come in contact with the hard flocked surfaces.

10. The medicated flocked article of claim 3, wherein the medicated flocked article protects an article from odor, insect, pest, fungi, bacteria, and virus.

11. The method of claim 8, wherein the fragrance is selected from a group consisting of: Fresh Floral SB, Musk GL, Cedar, Marine Ozone, Fruity TA, Lav.Fresh 101.

12. The method of claim 1, wherein the polymeric microcapsule comprising about 2% to 95% of beneficial agent, and about 1% to 30% of polymeric shell.

13. The method of claim 1, wherein the polymeric microcapsule has a mean diameter of about 1.0 microns to 500 microns.

14. The method of claim 1, wherein the polymeric microcapsule comprises a polymer forming the shell, which is impervious and substantially insoluble with water and the core material comprising beneficial agent.

15. The method of claim 14, wherein the polymer forming the shell of the polymeric microcapsule is selected from a group consisting of: polyethylenes, polyamides, polystyrenes, polyisoprenes, polycarbonates, polyesters, polyacrylates, polyureas, polyurethanes, polyolefins, polysaccharides, epoxy resins, vinyl polymers, and mixtures thereof.

16. The method of claim 14, wherein the polymer forming the shell of the polymeric microcapsule is impervious to the core and is reaction products of one or more amines with one or more aldehydes.

17. The method of claim 15, wherein the polymer of the polymeric microcapsule is formed by any of the combination: urea cross-linked with formaldehyde or gluteraldehyde, melamine cross-linked with formaldehyde, gelatin-polyphosphate coacervates cross-linked with gluteraldehyde, gelatin-gum Arabic coacervates, cross-linked silicone fluids, polyamine reacted with polyisocyanates, and mixtures thereof.

18. A method of preparing a hard flocked surface having beneficial feature, the method comprising:
premixing a polymeric microcapsule encapsulating a desired beneficial agent as a core material and water in 1:1 ratio in a high shear mixer for between 10 seconds to 30 minutes;
diluting by adding water to the premixed slurry to achieve a consistency of between 1 gms to 200 gms of polymeric microcapsules/liter of slurry;
re-circulating the slurry for between 1 minute to 100 minutes at a rate of between 100 gph to 6000 gph;
dipping, spraying or padding the flocked article into the slurry till it gets saturated with polymeric microcapsules; and
drying the flocked article by ambient air or dryer.

19. The method of claim 18, wherein the polymer microcapsules are applied to the flocked surface to achieve an application rate of between 0.1 to 1000 grams/square meter.

20. The method of claim 18, wherein the slurry is continuously agitated during the spray or dip application on a solid hard flocked surface.

21. The method of claim 18, wherein the spray, dip or padding application results in a uniform distribution of polymeric microcapsules over the solid hard flocked surface, without adversely affecting appearance.

22. The method of claim 18, wherein the suspension stabilizer is selected from a group consisting of one or more of: xanthan gum, carboxymethylcellulose, hydroxypropylcellulose, sodium polyacrylate, magnesium aluminum silicate, bentonite, lignin sulfonate, condensation product of sodium naphthalene sulfonate and formaldehyde, polyoxyethylenealkylaryl sulfates, polyoxyethylenestyrylphenyl ether sulfate and polyoxyethylenenonyl ether sulfate, and nonionic surface active agents such as polyoxyethylenenonylphenyl ether, polyoxyethylenestyrylphenyl ether, and polyoxyethylenealkylallyl ether can be admixed either singly or as a mixture of these substances.

23. The method of claim 18, wherein the wetting agent is selected from a group consisting of one or more of: nonionic surfactants such as sorbitan monoester (tween 20), alcohol ethoxylates and alkyphenolethoxylates and alkyl polyglucosides, anionic surf aceactive agents such as lignin sulfonate, condensation product of sodium naphthalene sulfonate and formaldehyde, polyoxyethylenealkylaryl sulfates, polyoxyethylenestyrylphenyl ether sulfate and polyoxyethylenenonyl ether sulfate, nonionic and anionic wetting agents used singly or as a mixture of these substances.

24. The method of claim 18, wherein the slurry comprises at least one formaldehyde scavenger.

25. A method for transferring beneficial agents to a hard flocked surface wherein the transfer is achieved by spraying or dipping the flocked surface in a slurry comprising:
a polymeric capsule containing a beneficial agent;
a suspension stabilizer;
a wetting agent; and
a chemical scavenger.

26. The method of claim 25 wherein the slurry is kept under sustained agitation during spraying, dipping, or padding operation.
